(19) United States
(12) Patent Application Publication
(10) Pub. No.: US 2021/0023976 A1
(43) Pub. Date: Jan. 28, 2021

(54) VEHICLE FOR PROVIDING A DIALYSIS SOLUTION

(71) Applicant: Fresenius Medical Care Deutschland GmbH, Bad Homburg (DE)

(72) Inventors: Klaus WOLF, Muedesheim (DE); Zdenek CERMAN, Idstein (DE); Stefan WEISS, Bad Homburg (DE); Moritz KEIM, Mainz (DE); Tim GLÄßER, Mainz (DE)

(73) Assignee: Fresenius Medical Care Deutschland GmbH, Bad Homburg (DE)

(21) Appl. No.: 16/971,368

(22) PCT Filed: Feb. 14, 2019
(86) PCT No.: PCT/EP2019/053683

§ 371 Date: Aug. 20, 2020

(30) Foreign Application Priority Data
Feb. 21, 2018 (DE) 10 2018 103 938.7

(51) Int. Cl. B60P 3/00; A61J 1/10; A61M 1/16; A61L 2/02; C02F 1/44; B60P 3/22; B60P 3/14

(52) U.S. Cl. CPC B60P 3/007; A61J 1/10; A61M 1/1656; A61L 2/022; C02F 1/441; C02F 2103/026; B60P 3/226; B60P 3/14; A61M 2209/045; A61M 2205/75; C02F 2303/04; B60P 3/2245

(57) ABSTRACT

The present invention relates to a power-driven land, water or air vehicle for providing dialysis solution receptacles, in particular bags, filled with dialysis solution, with the vehicle having a tank for receiving dialysis solution or for receiving a component of a dialysis solution, a storage region for the dialysis solution receptacles to be filled, and a filling station that is in fluid communication with the tank and that is configured to fill the dialysis solution receptacles with dialysis solution or with a component of the dialysis solution.

The present invention relates to a power-driven land, water, or air vehicle for providing dialysis solution receptacles, in particular bags, filled with dialysis solution.

It is known from the prior art that dialysis patients that have to carry out a peritoneal dialysis treatment attend a dialysis center to perform the treatment there. A known procedure furthermore comprises patients carrying out the treatment at home, which requires a supply of the patient with solution bags that contain the ready-to-use dialysis solution. The first-named variant is in particular difficult to implement for patients who have restricted mobility and who can therefore only attend a dialysis center with difficulty. The second variant is associated with logistical disadvantages that inter alia comprise the delivery of the solution bags to the patient and the storage of the solution bags at the patient's home.

Against this background, it is the underlying object of the present invention to provide a possibility by means of which the aforesaid disadvantages can be avoided.

This object is achieved by a vehicle having the features of claim 1.

Provision is accordingly made that the vehicle has a tank for receiving dialysis solution or for receiving a component of a dialysis solution, a storage region for the dialysis solution receptacles to be filled, and a filling station that is in direct or indirect fluid communication with the tank and that is configured to fill the dialysis solution receptacles with dialysis solution or with a component of the dialysis solution.

The vehicle can, for example, be a truck or bus, a ship, or also an aircraft, which is in particular sensible when very large distances to the patients have to be covered.

The vehicle has a tank in which one or more components are receivable or contained that are required for the preparation of a ready-to-use dialysis solution. To prepare the read-to-use dialysis solution, these components have to be mixed with further components of the dialysis solution, for which purpose further tanks or other receptacles are present in the vehicle which contain said further components. This mixing can take place manually or automatically.

It is pointed out that the present invention relates both to the vehicle per se, i.e. without the tank contents and any concentrates present, i.e. to the vehicle without the consumables such as water and concentrates required to prepare the dialysis solution, and includes the case that these materials are located in the vehicle, i.e. that the tank is filled and/or that the concentrate or concentrates are likewise arranged in suitable receptacles in the vehicle.

The dialysis solution is preferably a dialysis solution that is suitable and intended for the carrying out of a peritoneal dialysis treatment.

The preparation of the dialysis solution preferably takes place in a manner individual to the patient so that dialysis solutions tailored to the individual patient can be provided. This can in particular be implemented when RO water or water sterilized in a different manner is located in the tank and when an individual metering in of one or more ingredients or concentrates is made possible. This can take place in the filling station or also in the tank itself in that the concentrate or concentrates is/are supplied to the tank filled with water.

It is also possible that the ready-to-use dialysis solution is contained in the tank so that the addition of further components, in particular of concentrates, is not required.

The vehicle has a filling station in which the dialysis solution receptacles are filled with the dialysis solution, which is preferably ready to use, or with components of the dialysis solution, which is preferably ready to use.

The dialysis solution receptacles are preferably bags, i.e. dialysis solution receptacles having flexible walls. However, any other desired containers are also covered by the invention that only have rigid walls or that have a combination of flexible and rigid walls.

A ready-to-use dialysis solution can be filled into the dialysis solution receptacles at the filling station. It is, however, also conceivable that only some of a ready-to-use dialysis solution is filled into the dialysis solution receptacles at the filling station and the remaining components are added on the introduction of the dialysis solution into the patient. The remaining components can be located, for example, in cartridges or glass flasks, etc. that are added when the peritoneal dialysis patient is filled with the dialysis solution from the dialysis solution receptacle. It is, for example, conceivable that it is connected to the patient catheter, i.e. to the tube section that leads into the abdominal cavity of the patient and that is fixedly connected to the patient.

Provision is, however, preferably made that ready-to-use dialysis solutions are provided and are filled into dialysis solution receptacles by means of the vehicle, i.e. dialysis solutions that are introduced into the abdominal cavity of the patient without the addition of further substances.

It is achieved by the present invention that neither a complex delivery of dialysis solution receptacles filled with dialysis solution to the patient has to take place nor does the patient have to cover distances, that may be large, to collect the dialysis solution receptacles or to have a dialysis treatment performed.

The delivery of the filled dialysis solution receptacles takes place by means of the vehicle in accordance with the invention, e.g. directly to the residence of the patient or to his neighborhood, e.g. to a central location at which the patient can collect the dialysis solution receptacles.

The vehicle has a storage location or a store for storing dialysis solution receptacles to be filled. This store can be fillable or filled with dialysis solution receptacles, preferably bags, with the dialysis solution receptacles being able to be empty or being able to already contain one or more dialysis concentrates. The dialysis solution receptacles to be filled can be stackable to achieve a space-saving arrangement.

The dialysis solution receptacles can be designed as a strip-shaped row arrangement of dialysis solution receptacles between which desired break points are located at which the individual dialysis solution receptacles can be separated and are then available for filling.

The vehicle preferably has a sealing apparatus by means of which the dialysis solution receptacles are closed in a sterile manner after their filling.

It is conceivable that the vehicle has means for storing dialysis concentrate, with the filling station being directly or indirectly in fluid communication with the tank and with said means. A mixing of liquid located in the tank, which is preferably sterile water, with one or more concentrates thus takes place e.g. in the tank and/or in the dialysis solution receptacle and/or in the filling station itself in order to prepare the completed or substantially completed dialysis solution in this manner.

Provision is made in a preferred embodiment of the invention that the means are arranged relative to the tank such that the dialysis concentrate output from the means can be filled into the tank. In this case, dialysis concentrate is filled from a reservoir into the tank and is mixed therein. The mixing can, however, also only take place during the filling process in the filling station.

A mixing device such as an agitator that effects a complete intermixing and dissolution of the dialysis concentrate, that may be solid, can be located in the tank.

It is conceivable that different reservoirs for different dialysis concentrates are present so that an individualization of the dialysis solutions tailored to the patient or to a patient group is possible.

Adjustment means can furthermore be present by means of which the dialysis concentrate can be metered in a manner individual to the patient. These adjustment means can, for example, be pumps, valves, restrictors, or conveying means that e.g. add a specific quantity of the dialyzate to the tank, to the filling station, etc.

The vehicle can have a reading apparatus or another receiver by means of which it is possible to detect which dialysis solution is required. A conceivable embodiment of the invention thus comprises the patient communicating his identity or data, e.g. by a patient card or by means of a code, from which the solution to be dispensed results and which dialysis solution is to be prepared for this specific patient is stored in a memory of the vehicle or in an external memory. The devices for preparing the dialysis solution are then controlled accordingly, in particular one or more metering devices for adding the dialysis concentrate or concentrates so that the solution individual to the patient can be prepared.

In a further embodiment of the invention, the vehicle has a sterilization apparatus for sterilizing water, with the sterilization device preferably being an RO system. It is sufficient in this case to connect the vehicle to a mains water source from time to time. Sterile water that is used for the preparation of the dialysis solution that is filled into the dialysis solution receptacles is then prepared from the mains water by means of the RO system.

In general, however, other possibilities of sterilizing water are also covered by the invention.

The vehicle preferably has a filling port by means of which the tank can be filled from the outside. The filling can here, as stated, take place using mains water.

However, the filling with other liquids such as the filling with sterile water is also covered by the invention so that a sterilization device at the vehicle side can be dispensed with. It is also conceivable that the tank is filled with a dialysis solution that can be ready to use or that is ready to use after addition of one or more further ingredients.

It is conceivable that the tank is filled with sterile water.

It is furthermore conceivable that the vehicle has one or more concentrate containers that contain concentrate or concentrates that are to be mixed with water for the completed solution.

Provision can furthermore be made that the vehicle has a filling port and a sterilization device and that the tank is connected downstream of the filling port and of the sterilization system. After the filling in of water by means of the filling port, said water runs through the sterilization system such as an RO system and moves from there into the tank in the sterilized state.

The region of the vehicle in which the tank, the store for the dialysis solution receptacles to be filled and the filling station are located is preferably sterile.

The vehicle can have an output device for outputting the dialysis solution receptacles filled with the preferably ready-to-use dialysis solution that is preferably arranged at the outer side of the vehicle or in another, non-sterile, region of the vehicle. The patient can there collect the bags or other receptacles filled with fresh dialysis solution.

As stated above, the dialysis solution is preferably a peritoneal dialysis solution.

In a further preferred embodiment of the invention, the vehicle has a power supply that serves the operation of one of the components that is required for preparing the dialysis solution receptacles filled with the ready-to-use dialysis solution. It can be a battery or a rechargeable battery, a solar panel or a generator. These energy sources preferably supply the energy required for operating the system for providing dialysis solution receptacles even when the engine of the power-driven vehicle is switched off.

The vehicle can have a control that has to be activated by an operator such as in a conventional truck.

Provision can alternatively be made that the vehicle is an autonomously driving vehicle. It autonomously drives a specific route, with provision preferably being made that the route is calculated by the vehicle by suitable calculation means after the input or the presence of the addresses of the patients or of collection points.

It is furthermore conceivable that the vehicle has a memory that contains destination addresses of patients or of collection points that are to be driven to by the vehicle. A route having stops that is traveled through by interruption at individual stops is preferably fixed by means of a computer or on the part of the operator. Provision can be made here that a control is present that already prepares the dialysis solution receptacle or receptacles to be provided at the next stop before the reaching of a stop.

The vehicle can have one or more treatment stations for carrying out a dialysis treatment. The patient can supply the dialysis solution to himself by means of the patient catheter or can drain consumed dialysis solution from the abdomen into the drain at these treatment stations.

Provision is made in a further embodiment that the vehicle has a further tank for receiving non-sterile water or is filled therewith, with the further tank having a port for filling the tank and a fluid communication to the sterilization device.

The vehicle can thus be designed with one or more tanks.

The further tank of the vehicle can thus serve the reception of non-sterile water that is supplied to the sterilization apparatus and that can then be used for the preparation of the dialysis solution.

A concept results from this in that the vehicle does not necessarily have to have the total quantity of water on board at the start of the trip, but can rather top up the water levels during the tour. A stop can thus be made at different points during the tour to top up the starting tank with water. The water here can be non-sterile mains water that can, for example, also come from a domestic or garden connection for water. Sterile water can thus be generated during the tour. The RO system is fed from the water from the starting tank and conducts the purified water into the tank for purified water, the so-called storage tank.

It is furthermore conceivable for the port for filling the reception tank or the further tank to include a filter element. A particulate filter or the like that ensures a pre-purification of the mains water is e.g. conceivable.

There is yet a further tank that serves as a mixing container in a further preferred embodiment. In such an embodiment, the tank for receiving purified water, the storage tank, is not used for mixing the dialyzate, but rather only for receiving purified water. The purified water is conducted into the further tank, the mixing tank, and is mixed with a concentrate there to form a ready-to-use dialysis solution.

The present invention furthermore relates to a method of providing dialysis solution receptacles filled with dialysis solution using a vehicle in accordance with one of the claims 1 to 17. The method can comprise one or a plurality of the above-named steps and/or one or more of the measures described in claims 1 to 17.

It is pointed out at this point that the terms “a” and “one” do not necessarily refer to exactly one of the elements, even though this represents a possible embodiment, but can also designate a plurality of elements. The use of the plural equally also includes the presence of the element in question in the singular and, conversely, the singular also includes a plurality of the elements in question.

Further details and advantages of the invention will be explained in more detail with reference to an embodiment shown in the drawing.

There are shown:

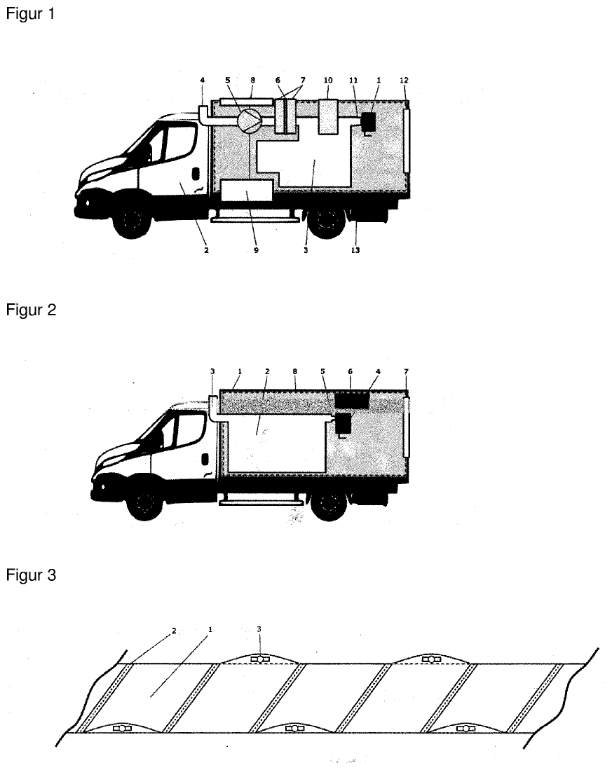

FIG. 1: a schematic sectional unit through a vehicle in accordance with the invention with a sterilization apparatus;

FIG. 2: a schematic sectional unit through a vehicle in accordance with the invention without a sterilization apparatus;

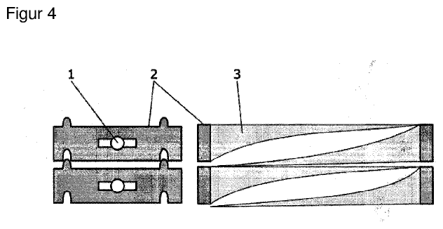

FIG. 3: a perspective view of a strip of bags separable from one another for receiving the dialysis solution; and

FIG. 4: a side view and a front view of two stacked bags of the strip in accordance with FIG. 3.

FIG. 1 shows a small truck 2 as a vehicle in accordance with the invention in a schematic longitudinal sectional view.

A storage location or a store for the bags or other dialysis solution receptacles is shown by reference numeral 1.

The tank 3 contains sterile water. Water such as mains water is filled into the vehicle via the filling port 4 and moves from there by means of the pump 5 into a membrane system 6 and into a filter system 7. These systems are components of a reverse osmosis (RO) system by means of which sterile water that is stored in the tank 3 is prepared from the non-sterile mains water.

Reference numeral 10 marks a concentrate container that contains dialysis concentrate in a solid or liquid form.

At least two such concentrate containers are preferably provided to be able to prepare different dialysis solutions or dialysis solutions individual to a patient.

Depending on the desired composition or concentration of the dialysis solution, dialysis concentrate is filled from the concentrate container or containers 10 into the tank 3 and is mixed with or dissolved in the sterile water therein.

Once a complete mixing/dissolving of the concentrate with/in the water has taken place, the filling of the bags can begin. For this purpose, the bags are moved out of the store to the filling station 11 and are there mixed with the solution located in the tank and are subsequently sealed in a fluid-tight manner.

The filled dialysis solution receptacles move from the filling station 11 to the output device 12 that is located at the rear part of the vehicle in the embodiment and at which they can be accepted by the user.

The dashed line 13 marks the sterile region of the vehicle. Since the bags and the dialysis solution are located in the sterile region, a sterilization of the bags after their filling is no longer necessary. The output device 12 represents the interface between the sterile region and the environmental atmosphere.

One or more of the aforesaid parts can be replaceable. This in particular applies to the filter 7, to the membrane 6, and to the concentrate reservoir 10.

To ensure a power supply for the components required within the framework of the preparation of the dialysis solution, the vehicle can be equipped with solar panels and/or a battery and/or a rechargeable battery 8 and/or with a generator 9. These elements enable the power supply even if the engine of the vehicle 2 is switched off.

FIG. 2 shows an embodiment in which the vehicle does not have a sterilization apparatus for water.

The vehicle 1 has a tank 2 that is filled with sterile water or with the dialysis solution that contains all or at least some of the ingredients that are required for the ready-to-use dialysis solution.

The filling of the tank 2 takes place by means of a filling port 3.

The bags 4 to be filled are located in a store 6 in the vehicle and are filled with the dialysis solution from the tank 2 through the port or through the filling station 5. An output device 7 at which the bags filled with dialysis solution are provided is located at the rear end of the vehicle. This output device represents the transition between the sterile region (reference symbol 8) and the outer environment.

An individualization of the dialysis solution can be achieved in that the individual bags or other dialysis solution receptacles are prefilled with a liquid or solid dialysis concentrate or the concentrate individual to a patient is filled or conducted into the tank and/or to the filling station. This applies both to the embodiment in accordance with FIG. 1 and to that in accordance with FIG. 2.

It applies in general that either the tank can be filled or is filled with sterile water or that a sterilization device is present in the vehicle that carries out the sterilization of the filled-in (non-sterile) water.

The dialysis concentrate or one or more components of the ready-to-use dialysis solution can generally be present as solid or liquid.

The concentrate or the component or components of the dialysis solution can be located in the respective bag or dialysis solution receptacle and/or in the tank. They can then be added during the filling of the dialysis solution receptacles and/or on the inflow of the solution into the patient and can, for example, be present in cartridges, vials, ampoules, effervescent tablets, etc.

In the case that reusable elements are present, the vehicle can have a sterilization device for these elements such as for the tube or a tubing set, bags, etc.

In the event that the vehicle has a sterilization apparatus for the filled-in water, this is independent of specific filling stations for sterile water. There is thus only a need to top up the bags or other dialysis solution receptacles.

It is conceivable that the vehicle has connectors that are directly connectable to the patient catheter. The first inflow volume can thereby be administered, for example, without a bag being necessary for this purpose. The further treatment then takes place using bags that are manufactured in the vehicle and that the user can take home.

If distillation is used as the sterilization method, a power consumption can be approximately assumed of 750 W per liter, with approximately 4 liters an hour being prepared.

The reverse osmosis has a power consumption of approximately 50 W per liter, with a rate of 20 liters an hour being able to be assumed.

The vehicle can drive by operator control or autonomously. It is conceivable that a driver having medical knowledge is used to be able to provide aid on the use of the dialysis solutions. The vehicle can likewise be a ship or a bus in which a waiting room and/or a treatment room is provided.

FIG. 3 shows a strip of mutually connected bags 1 that are releasable from one another along the desired break points 2. Reference numeral 3 marks connectors that are arranged, as can be seen from FIG. 3, at alternating sides of the bags 1. The space requirements for the stacked bags can be reduced in this manner. A further space saving results in that the connector does not project, but is rather likewise flat or planar.

As can be seen from FIG. 4, the connector 1 does not project, or only projects negligibly, over the stackable side edges 2 of the bags 3. To secure the stack, the side edges 2 have projections and recesses into which the projections engage and thus secure the stack.

As stated above, provision is preferably made that the patients have to authenticate themselves e.g. by a patient card or in another manner to obtain one or more bags filled with dialysis solution. This authentication also contains data on the desired composition of the dialysis solution so that once the authentication has taken place, the metering of the concentrate or the preparation of the solution can take place in a manner individual to the patient.

1. A power-driven land, water or air vehicle for providing dialysis solution receptacles, in particular bags, filled with dialysis solution, with the vehicle having a tank for receiving dialysis solution or for receiving a component of a dialysis solution, a storage region for the dialysis solution receptacles to be filled, and a filling station that is in fluid communication with the tank and that is configured to fill the dialysis solution receptacles with dialysis solution or with a component of the dialysis solution.

2. A vehicle in accordance with claim 1, characterized in that the vehicle has a closure apparatus for the fluid-tight closing or sealing of the filled dialysis solution receptacles.

3. A vehicle in accordance with claim 1, characterized in that the vehicle has means for storing dialysis concentrate, with provision preferably being made that the filling station is in fluid communication with the tank and with said means.

4. A vehicle in accordance with claim 3, characterized in that the means are arranged relative to the tank such that the dialysis concentrate dispensed from the means can be filled into the tank.

5. A vehicle in accordance with claim 3, characterized in that adjustment means are present by means of which the dialysis concentrate can be metered in individual to a patient.

6. A vehicle in accordance with claim 1, characterized in that the vehicle has a sterilization device for sterilizing water, with the sterilization device preferably being an RO system.

7. A vehicle in accordance with claim 1, characterized in that the vehicle has a filling port by means of which the tank can be filled from the outside.

8. A vehicle in accordance with claim 1, characterized in that the tank is filled with sterile water; and/or in that the vehicle has a filling port and a sterilization device, with the tank being connected downstream of the filling port and the sterilization system.

9. A vehicle in accordance with claim 1, characterized in that the vehicle has means for storing dialysis concentrate, with the means being formed by the dialysis solution receptacles, by the tank, or by separate concentrate receptacles.

10. A vehicle in accordance with claim 1, characterized in that the vehicle has an output device for outputting the dialysis solution receptacles filled with the dialysis solution that is preferably arranged at the outside of the vehicle.

11. A vehicle in accordance with claim 1 characterized in that the vehicle has a power supply that serves the operation of one of the components that are required for providing the dialysis solution receptacles filled with the dialysis solution.

12. A vehicle in accordance with claim 1 characterized in that the vehicle has a control that is to be actuated by an operator; or in that the vehicle is an autonomously driving vehicle.

13. A vehicle in accordance with claim 1, characterized in that the vehicle has one or more treatment stations for carrying out a dialysis treatment.

14. A vehicle in accordance with claim 1, characterized in that the vehicle has a memory that contains destination addresses of patients or of collection points that are to be driven to by the vehicle.

15. A vehicle in accordance with claim 6, characterized in that the vehicle has a further tank for receiving non-sterile water or is filled therewith, with the further tank having a port for filling the tank and having a fluid communication with the sterilization device.

16. A vehicle in accordance with claim 15, characterized in that the port for filling the tank or the further tank includes a filter element.

17. A vehicle in accordance with claim 15, characterized in that the vehicle has a third tank that is in fluid communication with the further tank and serves as a mixing container for mixing the sterile water with one or more concentrates.

18. A method of providing dialysis solution receptacles filled with dialysis solution using a vehicle in accordance with claim 1.
